(19) United States
(12) Patent Application Publication
(10) Pub. No.: US 2007/0265521 A1
(43) Pub. Date: Nov. 15, 2007

(54) Integrated MRI and OCT system and dedicated workflow for planning, online guiding and monitoring of interventions using MRI in combination with OCT

(76) Inventors: Thomas Redel, Poxdorf (DE); Estelle Camus, Erlangen (DE); Oliver Meissner, Munchen (DE)

Correspondence Address: BRINKS HOFER GILSON & LIONE, P.O. BOX 10395, CHICAGO, IL, 60610, US

(21) Appl. No.: 11/434,540

(22) Filed: May 15, 2006

(51) Int. Cl. A61B 5/055

(52) U.S. Cl. 600/411; 600/476

(57) ABSTRACT

A system and method for an integrated imaging system with magnetic resonance imaging (“MRI”) and optical coherence tomography (“OCT”). The system includes an MR scanner and OCT device configured to generate images. The system is further operative to assist a clinician in intravascular interventions, endoluminal interventions, biopsies and other guiding or therapy monitoring processes utilizing the imaging of MR and OCT.

### BACKGROUND

The present disclosure relates to an integrated magnetic resonance imaging (“MRI”) and optical coherence topology (“OCT”) system.

Atherosclerotic vascular disease is the underlying pathology for ischemic heart disease, peripheral arterial disease and strokes and is a leading cause of death and disability in many countries. Atherosclerosis is a disease affecting the arterial blood vessels and is frequently caused by the formation of plaque in the arteries. The plaque in the arteries can result in blockage or decreased blood flow. A decrease in the blood supply to an organ, tissue, or other body part caused by constriction or obstruction of the blood vessels is a common medical condition with very serious consequences, such as a heart attack. After a determination of the narrowing or blockage in the coronary or peripheral arteries, a common therapeutic approach is angioplasty. Angioplasty uses a balloon-tipped catheter to unblock a vessel, or repair the vessel through replacement of a part of the vessel. A stent may be used in the vessel to expand the size of the vessel. One example approach is percutaneous transluminal angioplasty (“PTA”) with or without stent implantation.

New vascular approaches, like cryotherapy, atherectomy, or the use of sophisticated new stent designs, such as drug eluting or bioabsorbable stents are gaining more importance. Surgical interventions can often be replaced by newer interventional procedures. Interventional procedures allow for either diagnosis or treatment by accessing the body through a cut or hole. One example is the insertion of a tube or catheter into a blood vessel. Alternatively, an interventional procedure may include the use of electromagnetic radiation. However, the interventional procedures may result in increased time and expense versus surgical procedures.

Interventional angiography currently serves as the most common procedure for interventional cardiologists/radiologists regarding treatment, planning, and interventional guidance. Angiography is an x-ray image of arteries or veins to detect a blockage or potential blockage. Angiography has been used as a two-dimensional imaging modality within a vessel for the planning, guiding and controlling of interventional procedures. Specifically, angiography may be used for determining the length and diameter of a vessel segment to be treated and for selection of a treatment device.

With the advent of newer imaging techniques like magnetic resonance (“MR”) or MRI angiography, conventional angiography may be displaced for diagnostic reasons. Angiography has limitations such as the use of radiation, which may cause complications to the patient. One problem is caused by the fact that bioabsorbable stents like magnesium based stents cannot be visualized with fluoroscopy due to their low atomic number. Additionally, iodinated contrast agents used in angiography may cause allergic reactions and are potentially harmful. The contrast agent or dye that is injected into an artery and flows through the arteries to improve an image taken of the artery such as with an X-ray. The contrast agent provides an improved image of by increasing the contrast of the image in the desired areas, such as specific arteries. This technique may be used for major arteries and those arteries connected to it.

However, in complex cases, invasive imaging techniques like intravascular ultrasound (“IVUS”) may be used to get more detailed information on the exact geometry of the stenosis, to plan treatment, and to aid in the exact placement of a stent. However, IVUS may be expensive to the point of only being practical as a research tool, rather than a clinical technique. In addition, IVUS is of limited spatial resolution, which may not allow for accurate assessment of the placement of the stent with respect to the vessel wall. In order to ensure the complete apposition of stent struts to a vessel wall to avoid the blocking of the vessel, accurate stent placement is very important. However, IVUS may not allow for exact placement control due to artifacts caused by the stent material.

Magnetic Resonance Imaging (“MRI”) offers an ability to obtain information on the vascular tree combined with high-resolution imaging of the vessel wall without the use of radiation or iodinated contrast material. Real-time imaging and interventional procedures with Magnetic Resonance (“MR”) have been enabled with ultrafast MR sequencing. Newer generation MR scanners, like the MAGNETOM ESPREE, support ultrafast MR with a significant reduction to the magnetic core and larger gantry sizes resulting in better patient access. However, MRI does have some drawbacks. First, MRI can only be used with restrictions on anyone who has any metal implants in their body. Screws, plates, or artificial joints in the area of a scan can cause distortions of the images and may be harmful to the patient. Second, MRI sequences with submillimeter resolution are time consuming and may be prone to artifacts caused by patient movement. Third, MRI does not provide the resolution necessary to differentiate atherosclerotic plaque components or infiltration of tumor with adequate tissue characterization.

Optical Coherence Tomography (“OCT”) is a light-based imaging modality capable of providing histology-like information of the vessel wall and atherosclerotic plaques. OCT allows for differentiation of different tissue types in plaques, in inflammation as well as in cancerous tissue. OCT allows for quantification of luminal geometry and guiding of interventional procedures in real-time. OCT is generally used for ophthalmology purposes, but more recently has been used for tissue imaging. In new catheter-based systems, OCT may be used for intravascular as well as intraluminal use. Therefore, examination of atherosclerotic plaques will be possible. Even in so called “vulnerable plaques,” the differentiation of the fibrous cap and the lipid core is possible for the first time. Due to limited susceptibility to metallic artifacts, OCT may allow control of exact placement of different stent types. Even bioabsorbable stents are visible to OCT.

### BRIEF SUMMARY

By way of introduction, the embodiments described below include a system and method for combining Magnetic Resonance (“MR”) with Optical Coherence Tomography (“OCT”) to develop a system for performing MR guided interventional procedures.

In a first aspect, a system for imaging includes a processor and a user interface coupled with the processor. The user interface is configured to control at least a part of the system. A magnetic resonance (“MR”) scanner is coupled with the processor and configured to generate a first image. An optical coherence tomography (“OCT”) device is coupled with the processor and configured to generate a second image. A display is coupled with the processor and configured to display at least one of the first image, the second image, or combinations thereof. The processor is configured to combine the first image and the second image to generate an overlaid image.

In a second aspect, an integrated magnetic resonance (“MR”) and optical coherence tomography (“OCT”) system for imaging includes a user interface. The user interface is configured to control at least a part of the system. An MR scanner and an OCT device are operable in response to a user interface. The MR scanner is configured to generate a first image and the OCT device is configured to generate a second image.

In a third aspect, a method of utilizing an integrated magnetic resonance (“MR”) imaging and optical coherence tomography (“OCT”) imaging system includes identifying a target area. An OCT catheter is inserted in the target area. The target area is characterized based on OCT data gathered from the OCT catheter. The catheter is guided with the MR imaging.

In a fourth aspect, a method of utilizing an imaging system includes integrating, at least in part, a magnetic resonance (“MR”) imaging system and an optical coherence tomography (“OCT”) imaging system. A user interface is provided for guiding an OCT catheter with MR imaging.

The present invention is defined by the following claims, and nothing in this section should be taken as a limitation on those claims. Further aspects and advantages of the invention are discussed below in conjunction with the embodiments.

### BRIEF DESCRIPTION OF THE DRAWINGS

The components and the figures are not necessarily to scale, emphasis instead being placed upon illustrating the principles of the invention.

FIG. 1 is a block diagram showing one embodiment of a system integrating MRI and OCT;

FIG. 2 is a block diagram showing a second embodiment of a system integrating MRI and OCT;

FIG. 3 is a diagram showing an embodiment of an arrangement of the integrated MRI and OCT system;

FIG. 4 a flowchart diagram showing one embodiment of a workflow using an embodiment of the integrated MRI and OCT system;

FIG. 5 is a flowchart diagram showing another embodiment of a workflow using an embodiment of the integrated MRI and OCT system; and

FIG. 6 is a flowchart diagram showing another embodiment of a workflow using an embodiment of the integrated MRI and OCT system.

### DETAILED DESCRIPTION OF THE DRAWINGS AND THE PRESENTLY PREFERRED EMBODIMENTS

With the optimization of magnetic resonance (“MR”) scanners and sequences as well as with the innovation of MR compatible interventional devices, MR will become a common choice for complex cardiovascular interventions. Conducting complex interventional procedures under MR guidance has advantages to both, the patient and the doctors due to the lack of radiation and the lack of iodinated contrast material. Optical coherence tomography (“OCT”) offers histology-like cross-sectional information of vessel walls and luminal dimensions. In addition, OCT may be used near or inside an MR scanner without causing metallic artifacts or a reduction in signal intensity. Finally, OCT can be used to visualize bioabsorbable Magnesium (“Mg”) stents. The combination of MRI and OCT may lead to a better placement of such devices with possible better long-term results.

Another important advantage of OCT is the ability to use a fiberoptic imaging probe inside a Magnetic Resonance (“MR”) scanner without the problems of incompatibility due to metallic components. MR scanners cannot be used near any metallic substance. Typical imaging probes may include at least some metallic components which makes them incompatible with MR imaging. Fiber-optic imaging probes do not include metallic components and can be used in conjunction with an MR scanner. A combination of MR and OCT imaging provides a useful means for planning, conducting and monitoring of cardiovascular interventional procedures that may lead to an improved workflow without the need of radiation or iodinated contrast material.

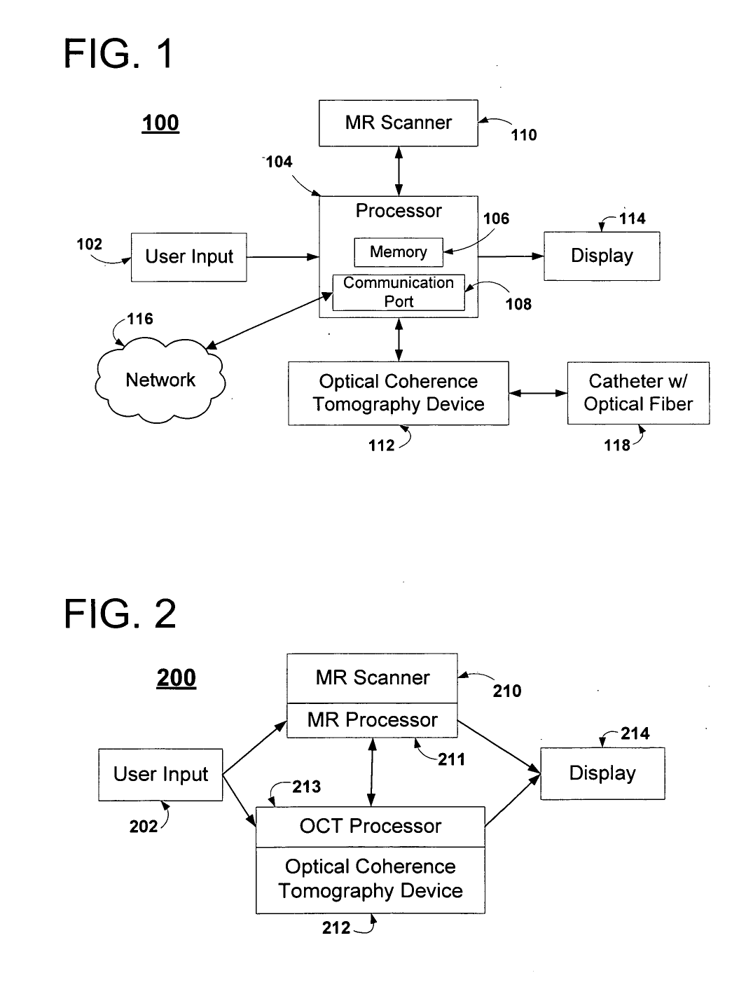

FIG. 1 is a block diagram showing one embodiment of a system 100 integrating MRI and OCT. The system 100 includes a processor 104 coupled with a memory 106 and a communication port 108. The processor receives input from a user input 102 and is further coupled with a network 116. A magnetic resonance (“MR”) scanner 110 and an Optical Coherence Tomography (“OCT”) device 112 are coupled with the processor 104. A display 114 is coupled with the processor 104 to output the results of the system 100. Additionally, an optical fiber catheter 118 may be used with the OCT device 112. The user input 102 and display 114 provide a user interface for controlling and/or viewing results from the system 100.

Additional, different or fewer components may be provided. The system 100 may include a personal computer, workstation, network, or other now known or later developed system for processing data. In one embodiment, the system 100 includes an MR scanner 110 and an OCT device 112 coupled with or including the processor 104. The MR scanner 110 and OCT device 112 are integrated and operative to create an image or produce data. Accordingly, the system 100 may be a computer aided imaging system for medical diagnosis, treatment, or another medical procedure.

The MR scanner 110 may be operative to display or record MR images. MRI uses a strong magnetic field to generate 2D or 3D images. MR scanners allow for a cross-sectional imaging of the inside of a living organism. MRI is harmless to the patient and it does not utilize ionizing radiation. The MR scanner 110 may be used to image the arteries with magnetic resonance angiography. Alternatively, interventional MRI uses the images produced by the MR scanner 110 to guide an invasive or non-invasive interventional procedure, such as insertion of a probe or stent. In system 100, the MR scanner 110 generates 2D or 3D images of a target area on an object. The target area may be a cancerous tissue, an inflammation area, a blood vessel, or arteries of a patient.

The OCT device 112 may be operative to display or record images. OCT is a light-based imaging modality that can provide histology-like information about the vessel wall as well as different tissue types by providing images of the microscopic structure of the tissue. Either cross-sectional or sectional images are generated with ultra-high resolution. Current systems receive a resolution in the order of 4 to 20 μm, even submicron resolution is reported. Additionally, atherosclerotic plaques that may block or restrict the arteries can be viewed with OCT imaging. OCT allows for the identification of the condition of a blood vessel, such as its geometry and whether any plaque is present. Due to extensive scattering of the light signal used by red blood cells, the energy of light used in current OCT systems (600-1300 nm) is not able to see through flowing blood or blood clots. Therefore, an ischemic situation has to be created to obtain OCT images. This can be achieved by introducing a fiber-optic OCT probe, or catheter 118 (discussed below). In one embodiment, the OCT probe may only be 0.014 inches in diameter, and located inside a second combined flushing/occlusion catheter. Cross-sectional images may be generated by rotating a glass fiber, which may be located inside the catheter. An automated linear pullback of the glass fiber inside the catheter may allow for contiguous imaging of even longer vessel segments. The OCT device may be used for monitoring of invasive procedures, also in combination with a spectroscopic device or a fluorescence imaging device.

Additionally, OCT allows for guiding of interventional procedures in real-time. New stent devices, like bioabsorbable magnesium stents, can be visualized with OCT. Therefore, stent apposition to the vessel wall as well as stent symmetry and unfolding may be imaged better with OCT than with other imaging modalities. OCT may provide images of the growth of the endothelium (neointima) around a stent and the current condition of an implanted stent. The OCT device 112 provides the system 100 with images of a target area on an object, such as arteries of a patient.

According to one embodiment, the OCT device 112 and the MR scanner 110 are combined. In a combined embodiment, MR may be used to identify a target area inside a patient's body. This may be done in a non-invasive manner first. For a more specific diagnosis and treatment planning, more detailed information may be needed. In an interventional environment in the MR suite OCT may be used to obtain this information and/or to guide invasive treatment or biopsy. The OCT system may be placed inside or outside the MR suite. The MR suite houses the MR scanner and must be free of all metallic artifacts because the very strong magnetic field creating by an MR scanner. The physician performing the interventional procedure stands near the patient table inside the MR suite. In general, electrical equipment like user interfaces such as a joystick or displays need to be MR compatible. Because the space in the MR suite is limited, the MR and the OCT systems might share at least a processor, a common user interface and a display monitor. Nevertheless both systems may be operated independently.

The processor 104 may be a component in a variety of systems. For example, the processor 104 may be part of a standard personal computer or a PACs workstation. As another example, the processor 104 may be part of one of the imaging systems, such as the MR scanner 110 or the OCT device 112. The processor 104 may be one or more general processors, digital signal processors, application specific integrated circuits, field programmable gate arrays, servers, networks, digital circuits, analog circuits, combinations thereof, or other now known or later developed devices for analyzing and processing image data. The processor 104 may implement a software program, such as code generated manually (i.e., programmed). In one embodiment, the software may be configured for receiving, transmitting, developing, or analyzing images from either or both the MR scanner 110 and/or the OCT device 112.

The processor 104 may include or be coupled with a memory 106. The memory 106 is a computer readable storage media. Computer readable storage media may include various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one embodiment, the instructions are stored on a removable media drive for reading by an imaging system or a workstation networked with similar systems. An imaging system or work station uploads the instructions. In another embodiment, the instructions are stored in a remote location for transfer through a computer network or over telephone lines to the diagnostic system or workstation. In yet other embodiments, the instructions are stored within the imaging system on a hard drive, random access memory, cache memory, buffer, removable media or other device. In one embodiment, the memory 106 includes a random access memory for the processor 104. In alternative embodiments, the memory 106 is separate from the processor 104, such as a cache memory of a processor, the system memory, or other memory. The memory 106 may be operable to store imaging data from the MR scanner 110 and/or the OCT device 112.

The memory 106 is operable to store instructions executable by the processor 104. The instructions may include the operation and integration of imaging with the MR scanner 110 and the OCT device 112. The functions, acts or tasks illustrated in the figures or described herein are performed by the programmed processor 104 executing the instructions stored in the memory 106. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

In one embodiment, the memory 106 may include a database. The database may be a part of the memory 106, such that the memory 106 maintains a database configured to store data. The database may store any form or type of data. In one embodiment, the database stores imaging data related to diagnosis, treatment, analysis, or any medical procedure. The memory 106 may be an external storage device or database for storing recorded image data. Examples include a hard drive, compact disc (“CD”), digital video disc (“DVD”), memory card, memory stick, floppy disc, universal serial bus (“USB”) memory device, or any other device operative to store image data.

The system 100 may include a display 114 coupled with the processor 104 and configured to display imaging data stored in the memory 106. The display 114 may be a CRT, monitor, flat panel, plasma, LCD, projector, printer or other now known or later developed display device for outputting determined information. A user may view images on the display 114 created from the MR scanner 110 and/or the OCT device 112.

The display 114 may act as an interface for the user to see the functioning of the processor 104, or specifically as an interface with the software for creating images (e.g., configuring the MR scanner 110 and/or the OCT device 112). The data may also be stored in the memory 106.

In one embodiment, there may be a pluarality of displays for displaying images from the MR scanner 110 and the OCT device 112 on different displays. Alternatively, the display 114 may be unnecessary in an alternate embodiment. In one embodiment, it is possible to visualize either the MR or the OCT image. Alternatively, it is also possible to visualize both images on the same display 114 or at least to overlay the images. The processor 104 may combine an MR image and an OCT image to create an overlaid image. The overlaid image may be a combination of features from an MR image and an OCT image to improve image quality. In one example, the pixel data is combined to create an image with high resolution. The overlaid image may represent an averaging of multiple images.

The system 100 may further include a communication port 108. The communication port 108 may be a part of the processor 104 or may be a separate component. The communication port 108 may be created in software or may be a physical connection in hardware. The communication port 108 is configured to connect with a network 116, external media, the display 114, the MR scanner 110, the OCT device 112, or combinations thereof. The connection with the network 116 may be a physical connection, such as a wired Ethernet connection or may be established wirelessly as discussed below. Likewise, the additional connections with other components of the system 100 may be physical connections or may be established wirelessly.

The network 116 may include wired networks, wireless networks, or combinations thereof. The wireless network may be a cellular telephone network, an 802.11, 802.16, 802.20, or WiMax network. Further, the network 116 may be a public network, such as the Internet, a private network, such as an intranet, or combinations thereof, and may utilize a variety of networking protocols now available or later developed including, but not limited to TCP/IP based networking protocols. The network 116 may be used to communicate patient information or medical data regarding a patient. Images generated by the system 100 may be communicated through the network 116. In one embodiment, the display 114 is not needed because the output or the image data is transmitted over the network 116 to a different location, such as a hospital. Likewise, the user input 102 may be unnecessary if the user controls are received from the network 116 rather than the user input 102. In an alternate embodiment, the images from the system 100 may be posted on the Internet.

The user input 102 allows a user, such as a doctor, clinician, or medical technician to interact with the system 100. The user input 102 may include a keyboard, mouse, joystick, touch screen display or other device operative to interact with the system 100. The user input 102 gives a user control over the operation of the system, such as initiating and monitoring the MR scanner 110 and the OCT device 112. In addition, the user input 102 may allow the user the ability to guide a catheter for various applications using imaging from MR or OCT. The controls or functions of the MR scanner 110 and/or the OCT device 112 may be separate from or integrated in part or entirely into a common user interface of the system 100 or implemented with the processor 104 with the user input 102.

The system 100 may also include a catheter 118 coupled with the OCT device 112. A catheter may be inserted into a blood vessel or other body cavity. The catheter 118 is used for performing various procedures. The catheter 118 may be flexible for intraluminal applications inside a blood vessel, or may be rigid for virtual needle biopsies. In one embodiment, the catheter 118 may include special markers which can be detected by the MR scanner 110. The markers may be small coils at the distal tip, or at the balloon site. If the catheter 118 is for occlusion and/or flushing, the device may be placed in a target vessel under MRI control. The placement of a catheter 118 to a specific location is referred to herein as a target area for the catheter 118. The target area may include any location in which the catheter 118 is to be placed, but in specific examples may be a cancerous tissue, an inflammation area, a blood vessel, or an artery. The catheter 118 is connectable and detachable to the OCT device 112. The catheter 118 includes an optical waveguide, such as a fiber optic cable.

The catheter 118 with an optical fiber may be used in conjunction with the OCT device 112 for the development of OCT images. The probe or catheter is used with the OCT device 112 and can further be observed with the MR scanner 110. Likewise, OCT can be used to visualize bioabsorbable stents like Magnesium (“Mg”) stents, resulting in a better placement of such devices in the target area with possible better long-term results. Bioabsorbable stents may be used with a catheter 118 and placed at a target area using MR and OCT imaging. In alternate embodiments, as discussed above, the catheter 118 may allow for imaging, or may be an occlusion catheter.

FIG. 2 is a block diagram showing a second embodiment of a system 200 integrating MRI and OCT. The system 200 represents an alternate embodiment that is different from system 100 of FIG. 1. System 200 includes a user input 202 and a display 214 as in system 100. The MR scanner 210 is coupled with a MR processor 211 and the OCT device 212 is coupled with an OCT processor 213. The MR processor 211 and OCT processor 213 are coupled with one another and coupled with the user input 202 and the display 214. Additional, different or fewer components may be provided in system 200.

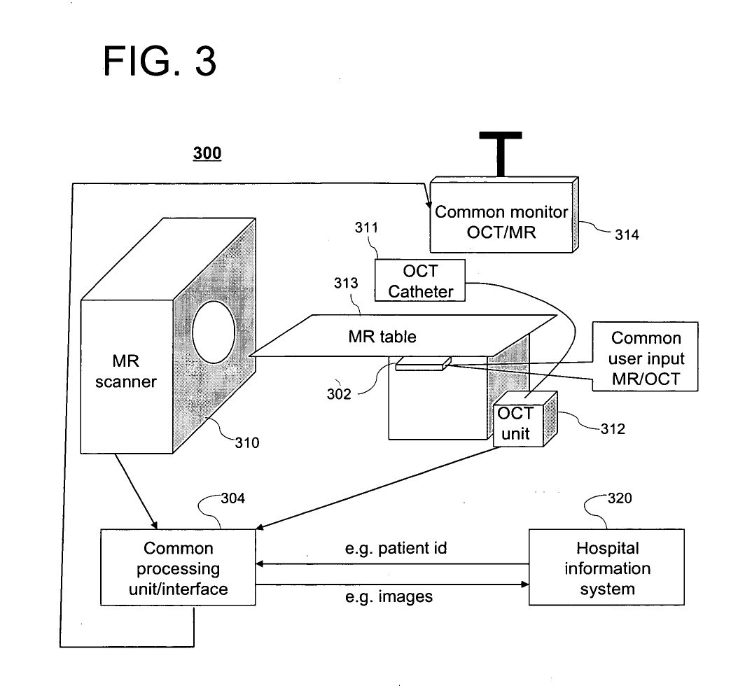

The system 200 in FIG. 2 represents an alternate embodiment in which the MR scanner 210 and the OCT device 212 have independent processors. System 100 in FIG. 1 and system 300 in FIG. 3 (discussed below) both show an embodiment in which a common processor is coupled with the MR scanner and OCT device. In system 200, the MR scanner 210 is coupled with an MR processor 211 and the OCT device 212 is coupled with the OCT processor 213. In one embodiment, the MR processor 211 and the OCT processor 213 are coupled with one another for sharing information such as patient or medical data as well as imaging information.

The user input 202 is similar to the user input 102 in FIG. 1. However, the user input 202 is coupled with both the MR processor 211 and the OCT processor 213. Likewise, the display 214 is similar to the display 114 in FIG. 1, except it receives output or image data from both the MR processor 211 and the OCT processor 213. The user input 202 and the display 114 provide a common or separate user interface for controlling the system 200. For example, the MR Scanner 210 and OCT device 212 operate independently with separate user interfaces. Not shown in system 200 may be a network, memory, communication port, catheter, or additional components.

FIG. 3 is a diagram showing an embodiment of the integrated MRI and OCT system 300. System 300 shows one implementation or setup of system 100. The system 300 includes an MR table 313 and a user input 302 coupled with an MR scanner 310 and an OCT unit 312. The MR scanner 310 and an OCT unit 312 are coupled with a common processing unit 304. The OCT unit 312 is further coupled with an OCT catheter 311. The processor 304 is coupled with a hospital information system 320 for transmitting patient information and outputted data. The outputted data or images may also be displayed on the common monitor 314.

The system 300 is one embodiment of an integrated MRI and OCT system. The MR scanner 310 and the OCT unit 312 are coupled with a common processing unit 304. The common processing unit is coupled with the common monitor 314, which displays the outputted images. A common user input 302 allows for user control of the system 300. The above mentioned components are similar to the embodiment that is shown in system 100 of FIG. 1; however, system 300 further includes an MR table 313. The MR table 313 receives a patient who is scanned by the MR scanner 310.

The common processing unit 304 is further coupled with the hospital information system 320 for transferring data. The hospital information system 320 includes patient information and medical data. The system 300 may provide documentation and archiving of the images of the MR scanner 310 and the OCT device 312. The OCT catheter 311 may be used to generate images from the OCT unit 312. The OCT catheter 311 is discussed above in conjunction with catheter 118 in FIG. 1. The processing unit 304 receives the image or images from the MR scanner 310 and/or the OCT device 312 and transmits that data to the hospital information system 320. In one embodiment, the transmission of the data is over a network. In addition, the hospital information system 320 may be used to provide patient information or medical data regarding a patient who is being imaged. The patient information may be used for the operation of the MR scanner 310 and/or the OCT device 312 on the patient. If the MR scanner 310 and the OCT device 312 have their own processors, then those processors may be configured to share patient data and medical information, as well as share or combine the images that are taken and generated by the user interface. During use, the patient is positioned on the table and in the MR scanner 310. The catheter connects with the OCT device 312. The catheter is within the patient and used for imaging while the patient is within the MR scanner 310.

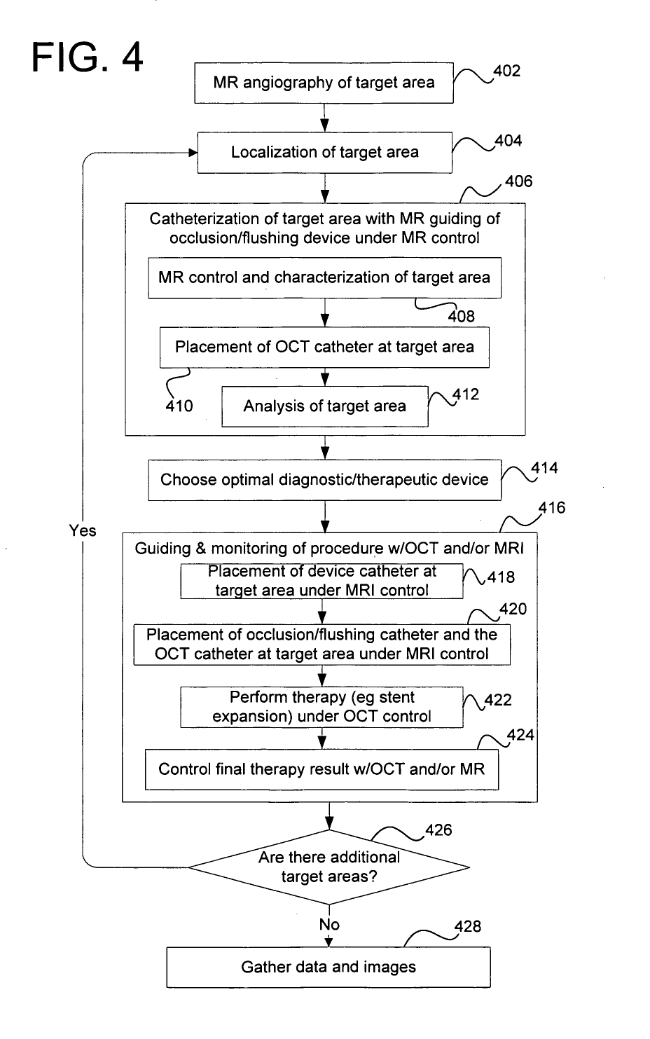

FIG. 4 is a flowchart diagram showing one embodiment of a workflow using an embodiment of the integrated MRI and OCT system. The process shown in FIG. 4 may utilize any of systems 100, 200, and/or 300 from FIGS. 1-3 or a different system. FIG. 4 represents a workflow related to an intravascular procedure inside blood vessels. The user interface is used to configure and operate the MRI and OCT devices. Controls or settings common to both types of imaging may be entered once. Alternatively, separate controls are provided using the same or separate input devices. Images are sequentially displayed. Where both types of imaging are being performed at a same time, adjacent images or a combination image are displayed. One or more images of one type may be persisted for continual display during generation of images of another type.

The combined or integrated system discussed above will allow for an optimized workflow. This optimized workflow may include different steps. In a first step, the target lesion is identified and localized under MR imaging. For that reason the user interface and display are related to MR. In a next step the occlusion balloon and the OCT imaging wire may be introduced into the patient body and positioned with respect to the target area. The movements of the catheter are monitored by simultaneous MR imaging. In a next step, OCT imaging may be performed including e.g. expansion of the balloon, flushing with saline. During this step, the user interface and display are used to control the OCT. Based on the detailed information given by OCT, treatment planning may be performed leading to the placement of a stent inside the vessel. The stent catheter is positioned inside the vessel under MRI control. The user interface and display are then used to control the MR. Before, during and after stent expansion, the position of the stent may be controlled under OCT and MR imaging. The display may be shared by MR and OCT or used by only one of these modalities.

Referring to FIG. 4, in step 402, 3D MR angiography of the targeted area is performed. The angiography may be enhanced with the use of a contrast agent. The target area may be a specific vessel, or may be intracerebral arteries, carotids, peripheral arteries, coronary arteries or veins, or bypass grafts. In step 402, the image is taken using MRI. In step 404, the target area is localized. Specifically, localization of a lesion to treat may be accomplished using high-resolution MR sequences.

In step 406, the target area is catheterized. In one example, the catheterization is of a target vessel using ultra fast MR sequences for online guiding and advancement of the catheter. Catheterization may involve inserting a tube (catheter) into a body cavity, or blood vessel. The catheter may be an occlusion, balloon, or flushing device inserted into the target area under MR control. MR control means that MR imaging is used to monitor the location of the catheter. Using the MR imaging, the catheter may be guided to the target area. Steps 408-412 are sub-steps of step 406.

In step 408, MR control is used for the characterization of the target area. The target area may be a lesion, and the characterization may be related to plaque composition, or quantification of lumen and vessel wall dimensions with OCT. In step 410, the placement of the OCT catheter may be for examining the target area under MR control. In step 412, the target area may be analyzed. The analysis may be of vessel geometry with dedicated software tools, ideally using multimodality monitors and tools integrated in the MR suite.

In step 414, an optimal diagnostic/therapeutic device is chosen based on the OCT data obtained in steps 410-414. The choice of an optimal therapeutic device may include a stent type, stent size, or balloon size.

In step 416, guiding and monitoring of the therapeutic procedure and post-therapeutic control is accomplished with OCT and/or MRI in real time. One example is visualization of stent deployment with OCT. This visualization may be advantageous for biodegradable stents. Steps 418-424 are sub-steps of step 416. In step 418, a device catheter is placed at the target area under MRI control. In step 420, an occlusion/flushing catheter and the OCT catheter are placed at the target area under MRI control. In step 422, a therapy may be performed under OCT control. One example of a therapy would be stent expansion. In step 424, the final therapy result may be controlled with OCT and/or MRI.

In step 426, if there are additional target areas, then the process returns to step 404 for localization of the additional target area. If there are no additional target areas, then in step 428, images and data are gathered. Specifically, final documentation includes control of the MR angiography, and archiving data and images.

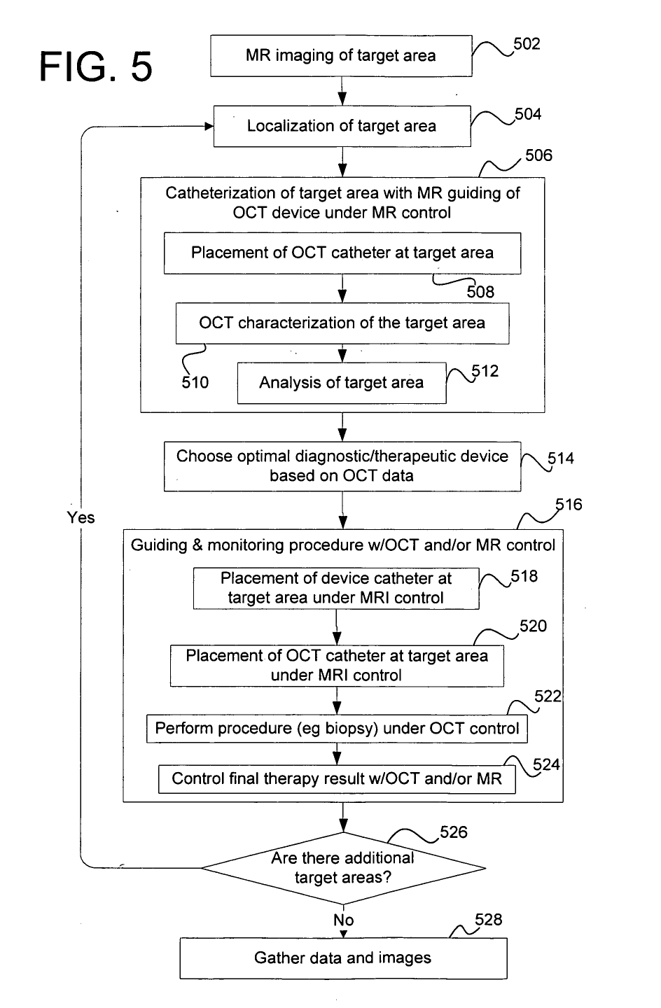

FIG. 5 is a flowchart diagram showing another embodiment of a workflow using an embodiment of the integrated MRI and OCT system. The process shown in FIG. 5 may utilize any of systems 100, 200, and/or 300 from FIGS. 1-3 or a different system. Specifically, the process illustrated by FIG. 5 relates to an endoluminal procedure. Possible procedures may include the examination of the hepato-billiary system, the gastro-intestinal tract or the uro-genital tract for early cancer detection, carcinoma in situ, or infiltration of the basal membrane.

In step 502, MR imaging of the targeted area is performed. The imaging may be 3D contrast enhanced MR imaging of the target area or target lumen. Examples of the lumen include the stomach, intestine, and spinal cord. In step 504, the target area is localized. The target are may include a lesion to be examined or treated using high-resolution MR sequences.

In step 506, catheterization of the target area is accomplished using ultra fast MR sequences for online guiding and advancement of an OCT device into the target area under MR control. Steps 508-512 may be sub-steps of step 506. In step 508, the OCT probe is placed in a location to examine the target area under MR control. In step 510, the target area is characterized using OCT. The characterization may include a cancer assessment, chronic inflammation, or quantification of lumen. In step 512, the target are may be analyzed. The online analysis may be of lumen geometry or tissue characterization with dedicated software tools, ideally using multimodality monitors and tools integrated in the MR suite.

In step 514, an optimal diagnostic or therapeutic device is chosen based on the OCT data. The therapeutic device may be a biopsy, local drug treatment, or ablation therapy.

In step 516, online guiding and monitoring of the therapeutic procedure and post-therapeutic control are accomplished with OCT in real time under MR control. Steps 518-524 are sub-steps of step 516. In step 518, a device catheter is placed at the target area under MRI control. In step 520, an OCT catheter is placed at the target area under MRI control. In step 522, a procedure may be performed under OCT control. One example of a procedure is a biopsy. In step 524, the final therapy result is controlled with OCT and/or MR.

In step 526 if there are additional target areas, then the process returns to step 504 for localization of the additional target area. If there are no additional target areas, then in step 528, images and data are gathered. Specifically, final documentation includes control of the MR imaging, and archiving data and images.

The combined usage of the MR and OCT types of imaging is performed efficiently with integrated control or output. For example, a user interface workflow is provided in software for stepping a user through the processes. The appropriate inputs are solicited and the desired outputs are generated. Alternatively, each type of imaging is controlled and outputs separately. The user manually integrates the processes.

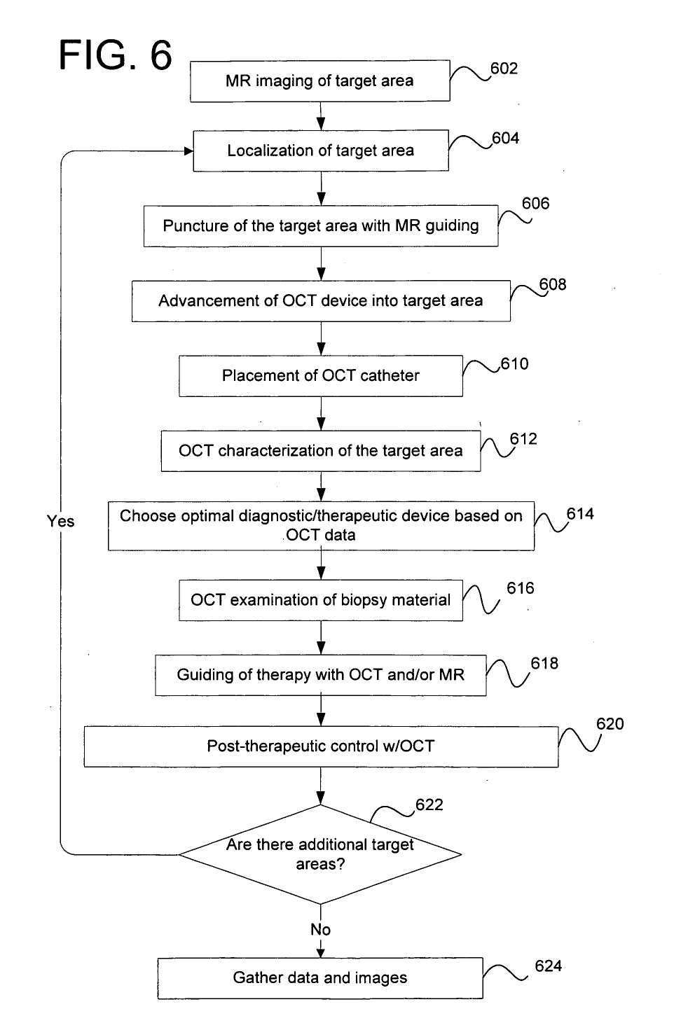

FIG. 6 is a flowchart diagram showing another embodiment of a workflow using an embodiment of the integrated MRI and OCT system. The process shown in FIG. 6 may utilize any of systems 100, 200, and/or 300 from FIGS. 1-3 or a different system. Specifically, the process illustrated by FIG. 6 relates to a needle based tissue characterization, biopsy guiding and therapy monitoring and control. A clinical example is the percutaneous puncture of liver tumors under MRI guidance and OCT detection of the biopsy site from inside the needle.

In step 602, MR imaging of the targeted area is performed. The imaging may be 3D contrast enhanced MR imaging of the target area. In step 604, the target area is localized. The target area may include a lesion to be examined or treated using high-resolution MR sequences. In step 606, the target area is punctured. The puncture at the target lesion may be accomplished using ultra fast MR sequences for online guiding. In step 608, the OCT device is advanced into the target area or target lesion. In step 610, the OCT catheter is placed into the target area for examination of the target lesion. In step 612, the target lesion is characterized using OCT. The OCT characterization of the target lesion may be accomplished using special molecular contrast agents.

In step 614, the optimal diagnostic or therapeutic device is chosen based on the OCT data. The device may be a biopsy needle or an ablation probe. The needle may be transparent to light in the 300 nm to 1500 nm range at the distal tip. In step 616, if the process is a biopsy, the biopsy material is examined. In step 618, the therapy is guided with OCT and/or MR. In step 620, the post-therapeutic control may be accomplished with OCT. For example, the control monitors tumor free borders, area of active inflammation, and/or therapeutic impact. In step 622 if there are additional target areas, then the process returns to step 604 for localization of the additional target area. If there are no additional target areas, then in step 624, images and data are gathered. Specifically, final documentation includes control of the MR imaging, and archiving data and images.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The above disclosed subject matter is to be considered illustrative, and not restrictive or limiting, and the appended claims are intended to cover all such modifications, enhancements, and other embodiments, which fall within the true spirit and scope of the present invention. Thus, to the maximum extent allowed by law, the spirit and scope of the present invention is to be determined by the broadest permissible interpretation of the following claims, including all equivalents, and shall not be restricted or limited by the foregoing detailed description.

To clarify the use in the pending claims and to hereby provide notice to the public, the phrases “at least one of <A>, <B>, . . . and <N>” or “at least one of <A>, <B>, . . . <N>, or combinations thereof” are defined by the Applicant in the broadest sense, superseding any other implied definitions hereinbefore or hereinafter unless expressly asserted by the Applicant to the contrary, to mean one or more elements selected from the group comprising A, B, . . . and N, that is to say, any combination of one or more of the elements A, B, . . . or N including any one element alone or in combination with one or more of the other elements which may also include, in combination, additional elements not listed.

1. A system for imaging comprising:
a processor;
a user interface coupled with the processor that is configured to control at least a part of the system;
a magnetic resonance (“MR”) scanner coupled with the processor and configured to generate a first image;
an optical coherence tomography (“OCT”) device coupled with the processor and configured to generate a second image; and
a display coupled with the processor and configured to display at least one of the first image, the second image, or combinations thereof;
wherein the processor is configured to combine the first image and the second image to generate an overlaid image.

2. The system according to claim 1, wherein the overlaid image improves the resolution and detail of the first image and the second image independently.

3. The system according to claim 1 further comprising:
a catheter coupled with the OCT device configured to record an image.

4. The system according to claim 3, wherein the catheter is optical and includes a special marker which can be detected by the MR scanner.

5. The system according to claim 4, wherein the catheter is guided using the MR scanner.

6. The system according to claim 1, wherein the OCT device is an invasive device configured to be in combination with a spectroscopic device or a fluorescence device.

7. The system according to claim 1 further comprising:
a communication port coupled with the processor; and
a network coupled with the communication port, wherein the at least one image is communicated over the network.

8. The system according to claim 7, wherein the user interface is located at a geographic location away from the MR scanner and OCT device, further wherein the control of the user interface is transmitted over the network to the system.

9. The system according to claim 1 further comprising:
a hospital information system that includes patient information and medical records.

10. The system according to claim 9, wherein the patient information is transmitted to the MR scanner and the OCT device.

11. The system according to claim 9, wherein the overlaid image is transmitted to the hospital information system.

12. An integrated magnetic resonance (“MR”) and optical coherence tomography (“OCT”) system for imaging, the system comprising:
a user interface;
an MR scanner coupled with the user interface; and
an OCT device coupled with the user interface;
wherein the MR scanner is configured to generate a first image and the OCT device is configured to generate a second image.

13. The system according to claim 12, wherein the at least one processor includes a first processor associated with the MR scanner and a second processor associated with the OCT device.

14. The system according to claim 13, wherein the first processor and the second processor are coupled with one another to share data.

15. The system according to claim 12, wherein the first image is combined with the second image to create an overlaid image.

16. The system according to claim 12 further comprising a display, the display configured to display the first image, the second image, an overlaid image of the first and second image, or combinations thereof.

17. The system according to claim 12 further comprising:
a communication port coupled with the processor; and
a network coupled with the communication port, wherein the at least one image is communicated over the network.

18. The system according to claim 17, wherein the user interface is coupled with the at least one processor over the network.

19. The system according to claim 12 further comprising:
a catheter coupled with the OCT device.

20. The system according to claim 19, wherein the catheter is optical and includes a special marker which can be detected by the MR scanner, further wherein the catheter is guided using the MR scanner.

21. A method of utilizing an integrated magnetic resonance imaging (“MRI”) and optical coherence tomography (“OCT”) imaging system, the method comprising:
identifying a target area using the MRI;
inserting and positioning an OCT catheter in the target area using the MRI; and
characterizing the target area based on OCT data gathered from the OCT catheter.

22. The method according to claim 21 further comprising inserting and positioning an occlusion or flushing catheter inside a vessel under MRI control before inserting and positioning the OCT catheter in the target area using the MRI.

23. The method according to claim 21 further comprising inserting and positioning a biopsy needle inside the target area under MRI control before inserting and positioning the OCT catheter in the target area using the MR.

24. The method according to claim 21 further comprising:
inserting an OCT device into a therapeutic device; and
monitoring a therapeutic procedure under OCT control.

25. The method according to claim 24, wherein the therapeutic procedure is at least one of a balloon dilatation, a stent expansion, a laser ablation, a local drug treatment, a treatment based on the application of temperature, a cryo-treatment, a photodynamic therapy, or combinations thereof.

26. The method according to claim 21, wherein the target area is one of a cancerous tissue, an inflammation area, a blood vessel, or combinations thereof.

27. A method of utilizing an imaging system, the method comprising:
integrating, at least in part, a magnetic resonance (“MR”) imaging system and an optical coherence tomography (“OCT”) imaging system; and
providing a user interface for guiding an OCT catheter with MR imaging.

28. The method according to claim 27, wherein the user interface is configured to control and position a therapeutic device using the integrated imaging system.

29. The method according to claim 28, wherein the therapeutic device is a bioabsorbable stent, a coated stent, or combinations thereof.
